(19) United States
(12) Patent Application Publication
(10) Pub. No.: US 2009/0088713 A1
(43) Pub. Date: Apr. 2, 2009

(54) METHOD OF REINFORCING A BOND BETWEEN WEB MATERIALS AND AN ABSORBENT ARTICLE COMPRISING BONDED WEB MATERIALS

(75) Inventor: Niclas Norrby, Goteborg (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/279,211

(22) PCT Filed: Feb. 13, 2006
(86) PCT No.: PCT/SE2006/000198

§ 371 Date: Oct. 15, 2008

(51) Int. Cl. A61F 13/56; B32B 37/00; B32B 38/00

(52) U.S. Cl. 604/365; 156/73.1; 156/290; 156/163

(57) ABSTRACT

A method of bonding a first and a second web material (2, 3) to each other, wherein the two web materials are brought together in the area to be bonded and thermal energy or ultrasonic energy is applied to at least one of the web materials in the area to be bonded. In order to improve the bonding strength an additional material (28) that is non-tacky at a temperature of 20° C. is applied to at least one of the web materials in at least a part of the area to be bonded prior to bringing the two web material (2, 3) together. In one aspect the web materials are components of a personal care absorbent article.

### TECHNICAL FIELD

The present invention refers to a method of bonding a first web material to a second web material by applying thermal energy or ultrasonic energy to at least one of the web materials in the area to be bonded. The invention especially refers to web materials forming part of a personal care absorbent article. One important application is pant-type absorbent articles wherein .the web materials form part of the body panels and the area to be bonded forms side seams of the pant-type absorbent article.

### BACKGROUND OF THE INVENTION

Personal care absorbent articles comprise articles like diapers, pant diapers, incontinence products, sanitary napkins, pantiliners etc. These articles comprise thin web material layers that are bonded to each other in selected areas. Some of these bonded areas incur significant stresses. This for examples applies to side seams of pant-type absorbent articles. Such articles are intended to be pulled up and down over the hips of a wearer to allow the wearer or caregiver to easily put on and remove the article when it has been soiled. In traditional pant diapers the cover material usually are made up from double layers of nonwoven materials. In the weld seams of this type of cover material, there are thus four layers of bonded nonwoven which usually gives a sufficiently strong weld seam.

It is further known to make pant-type absorbent articles having front and back body panels of an elastic web material, for example in the form of an elastic laminate comprising elastic threads or an elastic film sandwiched between layer of nonwoven material, such as spunbonded webs. The structure of the elastic laminate is adaped to give optimal elastic properties and soft and textile feel, at the same time as it should be thin, which may negatively effect the bond strength when bonding two elastic laminates together, such as in the side seams of a pant article, by ultrasonic bonding or thermobonding. Thus the bond strength, especially in the side seams, sometimes becomes insufficient for the stresses incurred during use of the article.

An example of an elastic laminate is described in WO 03/047488. This publication describes an elastic laminate composed of an elastic film applied between two thin nonwoven layers. During production of the elastic laminate, the nonwoven layers have been connected to the film, after which the film has been stretched until the bonds in the nonwoven layer breaks. The elasticity of the laminate is therefore made basically the same as the elasticity of the elastic film. An important disadvantage of this solution is that the weld seams between two elastic laminates of this kind have considerably less strength than the weld seams on said traditional diaper pants.

Other examples of elastic laminates are described in PCT/SE2004/001004, PCT/SE2004/001005 and PCT/SE2004/001415. In contrast to the production method according to WO 03/047488, the bonds of at least one of the nonwoven layers have not been completely broken open and, instead, the layer has a certain residual strength. However also with these types of elastic laminates the weld seams are weaker than in traditional diaper pants.

Further examples of absorbent articles which in part are made of elastic laminates are found in U.S. Pat. No. 6,476,289 and JP 10043235.

PCT/SE2005/000319 describes a hygiene pant, such as a diaper pant, comprising an elastic laminate and wherein the weld seams are reinforced by at least one nonwoven strip containing thermoplastic fibers. The elastic laminate has been welded together by ultrasonic welding with the nonwoven strip applied there between.

U.S. Pat. No. 6,837,961 discloses a pant diaper having tear resistant side seams comprising a combination of adhesive and an ultrasonic or thermal bond pattern. The application of adhesive however may cause process problems and product quality problems.

There is therefore still need for improvement of the bonding strength of bonds between certain web materials, for example elastic web materials, such as elastic laminates, in an efficient and simple way while maintaining a high product quality.

### OBJECT AND MOST IMPORTANT FEATURES OF THE INVENTION

An object of the present invention is to solve the above problem and to provide a cost efficient and simple method for improving the bonding strength of bonds between first and second web materials. The web materials are brought together in the area to be bonded and thermal energy or ultrasonic energy is applied to at least one of the web materials in the area to be bonded. Prior to bringing the two web materials together an additional material that is non-tacky at a temperature of 20° C. is applied to at least one of the web materials in at least a part of the area to be bonded. It is preferred that the additional material is already non-tacky at a temperature of 30° C., preferably also at 40° C. and more preferably also at 60° C. The problems of handling adhesive materials that are tacky also below their application temperature are herewith avoided. The additional material is applied at a temperature at which it is at least partly molten or softened so that it will adhere to the web material to which it is applied. The presence of the additional material in the area to be bonded improves the bonding strength of the bond between the two web materials.

In one aspect of the invention said additional material is applied by means of spraying.

In a further aspect the additional material is applied in the form of fibers or filaments.

In one embodiment the area to be bonded amounts to less than 10% of the total surface area of each of said first and second web materials.

In a further embodiment the area to be bonded has a defined length and width, wherein said width is no more than 30 mm, preferably no more than 25 mm and preferably no more than 20 mm.

According to one embodiment the web material comprises a polymeric material selected from polyolefins including polyethylene and polypropylene, polyesters, polyamides, copolymers and mixtures of such polymeric materials.

In a further embodiment the additional material comprises a polymeric material selected from polyolefins including polyethylene and polypropylene, polyesters, polyamides, copolymers and mixtures of such polymeric materials.

In one aspect of the invention said first and second web materials are components in a personal care absorbent article. In a still further aspect they form part of first and second body panels of a pant-type absorbent article and that said area to be bonded of said web materials intended to form a side seam of the pant-type absorbent article.

In one embodiment at least one of said first and second web materials comprises an elastic web material. In a further embodiment both the first and second web materials comprises an elastic web material. In a still further embodiment the first and/or second web material is an elastic laminate.

The elastic web material may be in a stretched condition during bonding. It may for example be stretched to a length which exceeds its untensioned length with at least 20% during bonding

In a further embodiment the additional material is applied to selected areas of said side seam.

In a still further embodiment the elastic web material has a basis weight of no more than 100 g/m², preferably no more than 90 g/m².

The elastic web material may be an elastic laminate comprising an elastic film applied between layers of fibrous material.

In one aspect of the invention the elastic web material constitutes the sole component of the first and second body panels at least in selected portions of the areas which are bonded together to form said side seams.

In a further aspect the tensile strength of the side seams (9, 10), in a direction transverse (x) to the side seam, is at least 5 N/25.4 mm, preferably at least 7 N/25.4 mm and more preferably at least 9 N/25.4 mm at least in the portions reinforced by the additional material (28).

In one embodiment the additional material is applied in an amount corresponding to a basis weight of at least 10 gsm, preferably at least 20 gsm and more preferably at least 30 gsm.

According to a further embodiment said elastic web material has an elasticity in the transverse direction of the article of at least 30%, preferably at least 50%, more preferably at least 70%, when measured according to the elasticity test specified in the description.

### BRIEF DESCRIPTION OF DRAWINGS

The invention will be described in the following in greater detail by way of example and with reference to the accompanying drawings, in which:

FIG. 1 shows a perspective view of a pant diaper.

FIG. 2 shows is a plan view of the pant diaper in its flat, uncontracted state prior to formation as seen from the body facing side.

FIG. 3 is a cross section according to the line III-III in FIG. 2.

FIG. 4 is a cross-section on an enlarged scale of an elastic laminate web material.

FIG. 5 and 6 illustrate schematically a method for measuring the tensile strength of a side seam of a pant diaper.

### DESCRIPTION OF PREFERRED EMBODIMENTS

The invention will in the following be closer described with reference to some embodiments shown in the accompanying drawings.

The drawings show an embodiment of a pant type absorbent article 1. Examples of such pant-type absorbent articles are pant diapers, sanitary pants and incontinence pants worn by incontinent adults. The pant-type absorbent article will below be referred to as a pant diaper. The term “absorbent article” refers to products that are placed against the skin of the wearer to absorb and contain body exudates, like urine, faeces and menstrual fluid. The invention mainly refers to disposable absorbent articles, which means articles that are not intended to be laundered or otherwise restored or reused as an absorbent article after use.

Said pant diaper typically comprises a front panel 2, a back panel 3 and a crotch portion 4. The article has a longitudinal direction y and a transverse direction x. The outer longitudinal edge portions 5 and 6 of the front panel 2, are connected by thermobonding or ultrasonic bonding to the respective longitudinal outer edges 7 and 8 of the back panel 3, to form side seams 9 and 10. A waist opening 11 and a pair of leg openings 12 and 13 are herewith formed.

The pant diaper comprises an outer coversheet covering the front 2 and back panels 3, said outer coversheet being in the form of an elastic web material 14, which is elastic at least in the transverse x-direction of the article. The elasticity in the x-direction may be at least 30%, preferably at least 50%, more preferably at least 70%, as measured by the elasticity test specified below.

Preferably the elastic web material is elastic also in the y-direction of the article. However the elasticity in the y-direction may be lower than in the x-direction. In one embodiment the elasticity in the y-direction is at least 20%.

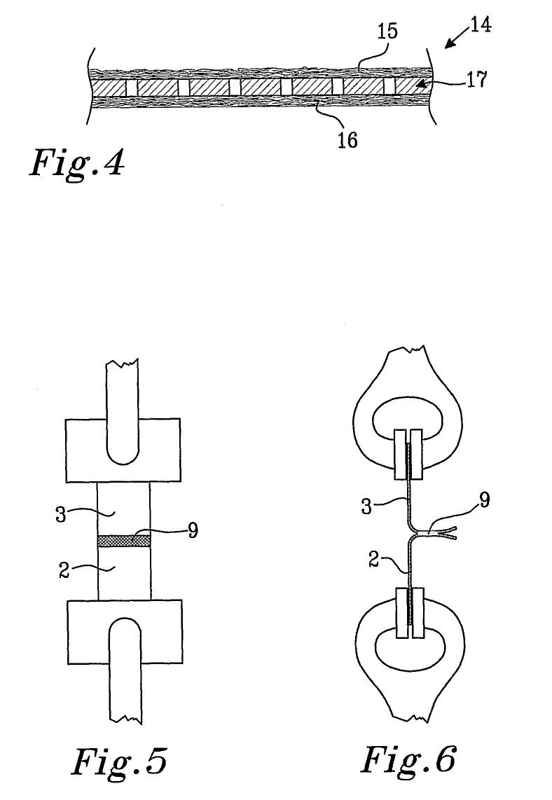

In the embodiment shown in FIG. 4 and described below the elastic web material 14 is an elastic laminate composed of first and second outer layers of fibrous material 15 and 16 and a middle elastic film layer 17 located between said fibrous layers. However it is understood that other types of elastic web materials may be used, such as elastic nonwoven materials, nonwoven materials which per se are inelastic, but which have been elastified by means of elastic threads etc. The elastic web materials may comprise one layer or two or more layers that have been laminated.

In the elastic laminate shown and described below it is preferred that the outer fibrous layers 15 and 16 are chosen so that they, in combination with the inner elastic film layer 17, give the material high resistance to puncture. They also provide a soft and cloth-like feel to the laminate. Examples of suitable materials are carded webs and spunbond materials. The basis weight of the fibrous material layers in the laminate should be between 10 and 35 g/m², preferably between 12 and 30 g/m², more preferably between 15 and 25 g/m². Examples of suitable polymers used in the fibrous materials are polyethylene, polyesters, polypropylene and other polyolefin homopolymers and copolymers. Natural fibres, for example cotton, may also be used as long as they provide the required properties. A mixture of polymers can contribute to a higher flexibility of the nonwoven layer, and through this, give the nonwoven material a higher elongation at maximum load. A mixture of polyethylene and polypropylene polymers has proved to provide good results in this respect. A mixture of fibers of different polymers is also possible.

The middle layer 17 is according to one embodiment of the invention an apertured elastic film having a basis weight between 20 and 80 g/m², preferably between 20 and 60 g/m². The film may be of any suitable elastic polymer, natural or synthetic. Some examples of suitable materials for the elastic film are low crystallinity polyethylenes, metallocene-catalyzed low crystallinity polyethylene, ethylene vinyl acetate copolymers (EVA), polyurethane, polyisoprene, butadiene-styrene copolymers, styrene block copolymers, such as styrene/isoprene/styrene (SIS), styrene/butadiene/styrene (SBS), or styrene/ethylene-butadiene/styrene block copolymer. Blends of these polymers may also be used as well as other modifying elastomeric or non-elastomeric materials. One example of a suitable film is an apertured three-layer elastomeric film of PE-SEBS-PE.

The total basis weight of the laminate is preferably 100 g/m²or less, more preferably no more than 90 g/m².

The elastic laminate 14 may be manufactured according to the method disclosed in WO 03/047488, wherein one spunbond layer 15 is applied to the film 17 in a tacky state and will thus bond to the film layer, while the other spunbond layer 16 is adhesively laminated to the film layer 17, using for example a pressure sensitive hot melt adhesive. Alternatively the laminate is manufactured according to a modified version of this known method, wherein the modification involves that the laminate is incrementally stretched (through intermeshing gears, IMG), to a point below the elongation at peak load of at least one of the non-elastic nonwoven layers to retain some strength for at least one of the nonwoven layers. The other layer may also be stretched to a point below its elongation at peak load, or to a point at which it will tear during stretching.

The method disclosed in WO 03/047488 involves stretching of the laminate above the point of failure of the fibrous material, so that the non-elastic layers break completely. Therefore, as described in WO 03/047488, the elongation of the laminate is not limited by the stretch modulus of the non-elastic material.

In a preferred embodiment at least one, preferably both fibrous layers, which are bonded to the elastic film, are not, in contrast to the method described in WO 03/047488, completely torn upon manufacture of a laminate according to the present invention. Selection of fibrous materials which have an elongation at maximum load greater than the elasticity of the elastic laminate allows the elastic film to stretch without being hindered by the fibrous layers. Such a selection also ensures that the fibrous layers contribute to the puncture resistance of the laminate, as they are not completely torn or broken during manufacture. Preferably both fibrous layers, or at least one of the fibrous layers have an elongation at maximum load that is at least 10% higher than the elasticity of the laminate. This is described in more detail in PCT/SE2004/001005, which is incorporated herein by reference.

The opacity of a material layer is the characteristic ability of the material layer to visually hide from view an underlying object or pattern. The opacity is measured in %, wherein 100% opacity means that nothing can be seen through the material layer and 0% means that the material layer is completely transparent. The opacity is measured by the Opacity Test disclosed in PCT/SE2004/001415.

Opacity of the laminate can be obtained by the incorporation of opacifying fillers into the laminate, particularly into the elastic film. Such pigments can be organic or inorganic dyes, colouring agents, or whitening agents. Inorganic materials such as titanium dioxide, inorganic carbonates, synthetic carbonates, talc, nepheline syenite, magnesium hydroxide, aluminium trihydrate siatomaceous earth, mica, natural or synthetic silicas, calcinated clays and mixtures thereof are preferred examples of opacifying fillers.

The filler is preferably added as a master batch at the extrusion of the film. One example of an appropriate concentration is about 5% filler by weight of the film.

It is further preferred that the elastic laminate 10 has a breathability (Water Vapour Transmission Rate) according to ASTM E96-00 Procedure D of at least 1500 g/m² 24 h, preferably at least 3000 g/m² 24 h.

The pant diaper according to the drawings have a core region 18 located in the crotch portion 4 of the article and extends a certain distance into the front 2 and back panels 3. The crotch portion 4 is herewith defined as the narrow part of the article intended to be worn in the wearer's crotch between the legs.

The pant diaper comprises a liquid permeable topsheet 19 and a liquid impermeable backsheet 20 covering the core region 18. An absorbent core 21 is enclosed between the topsheet 19 and the backsheet 20.

The liquid permeable topsheet 19 can consist of a nonwoven material, e g spunbond, meltblown, carded, hydroentangled, wetlaid etc. Suitable nonwoven materials can be composed of natural fibers, such as woodpulp or cotton fibres, manmade fibres, such as polyester, polyethylene, polypropylene, viscose etc. or from a mixture of natural and manmade fibres. The topsheet material may further be composed of tow fibres, which may be bonded to each other in a bonding pattern, as e.g. disclosed in EP-A-1 035 818. Further examples of topsheet materials are porous foams, apertured plastic films etc. The materials suited as topsheet materials should be soft and non-irritating to the skin and intended to be readily penetrated by body fluid, e.g. urine or menstrual fluid. The topsheet may be different in different parts of the absorbent article.

The liquid impervious backsheet 20 covering the core region 18 on the garment-facing side of the absorbent core 21 is of a liquid impervious material, such as a thin plastic film, e.g. a polyethylene or polypropylene film, a nonwoven material coated with a liquid impervious material, a hydrophobic nonwoven material, which resists liquid penetration or a laminate comprising plastic films and nonwoven materials. The core region backsheet material 20 may be breathable so as to allow vapour to escape from the absorbent core, while still preventing liquids from passing therethrough. Examples of breathable backsheet materials are porous polymeric films, nonwoven laminates from spunbond and meltblown layers, laminates from porous polymeric films and nonwovens. The backsheet 20 is preferably inelastic.

The absorbent core 21 can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times its weight and in an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly crosslinked to render the material substantially water insoluble. Preferred superabsorbent materials are further surface crosslinked so that the outer surface or shell of the superabsorbent particle, fiber, flake, sphere, etc. possesses a higher crosslink density than the inner portion of the superabsorbent. The superabsorbent materials may be in any form suitable for use in absorbent composites including particles, fibers, flakes, spheres, and the like.

A high absorption capacity is provided by the use of high amounts of superabsorbent material. For an absorbent core comprising a matrix of hydrophilic fibers, such as cellulosic fibers, and superabsorbent material, the proportion of superabsorbent material is preferably between 10 and 90% by weight, more preferably between 30 and 70% by weight.

It is conventional in absorbent articles to have absorbent cores comprising layers of different properties with respect to liquid receiving capacity, liquid distribution capacity and storage capacity. The thin absorbent bodies, which are common in for example baby diapers and incontinence guards, often comprise a compressed mixed or layered structure of cellulosic fluff pulp and superabsorbent polymers. The size and absorbent capacity of the absorbent core may be varied to be suited for different uses such as for infants or for adult incontinent persons.

The absorbent core may further include an acquisition distribution layer placed on top of the primary absorbent body and which is adapted to quickly receive and temporarily store discharged liquid before it is absorbed by the primary absorbent core. Such acquisition distribution layers are well known in the art and may be composed of porous fibrous waddings or foam materials.

The elastic web material 14 may cover the entire article, including the core region 18 and the entire front and back panels 2 and 3. However according to a preferred embodiment a substantial part of the crotch portion 4 of the article is free from the elastic web material 14. A “substantial part” used herein refers to at least 50%, preferably at least 75%.

A crotch portion web material 22, which preferably is a non-elastic nonwoven material, is arranged in the crotch portion 4 of the article and overlaps with the elastic front and back panels 2 and 3. The crotch portion web material 22 is joined in an overlapping manner to the front and back panels 2 and 3 respectively by means of ultrasonic welds 23, glue strings or the like.

An elastic waist band 24 is further provided which comprises a substantially non-elastic nonwoven material that is elasticized by elongate elastic members 25 such as elastic threads, contractably affixed between material layers, such as nonwoven materials. Elastic threads 26 may also be arranged around the leg openings 5 and 6 of the article. A strip of nowoven material 29 is further applied along the rear part of the leg opening area.

The liquid-impervious backsheet material 20 underlies the absorbent core 21 and adjacent areas immediately outside the absorbent core 21. The area covered by the liquid-impervious backsheet 20 is defined as the core region 18. The crotch nonwoven material 22 is arranged on the garment-facing side of the liquid-impervious backsheet 20 in the crotch portion 4 of the article. The core region 18 extends into the front and back panels 2 and 3 so that the elastic web material 14 and the liquid impervious backsheet 20 overlap in the outer parts of the core region 18, wherein the elastic web material 14 is arranged on the garment facing side of the liquid impervious backsheet 20.

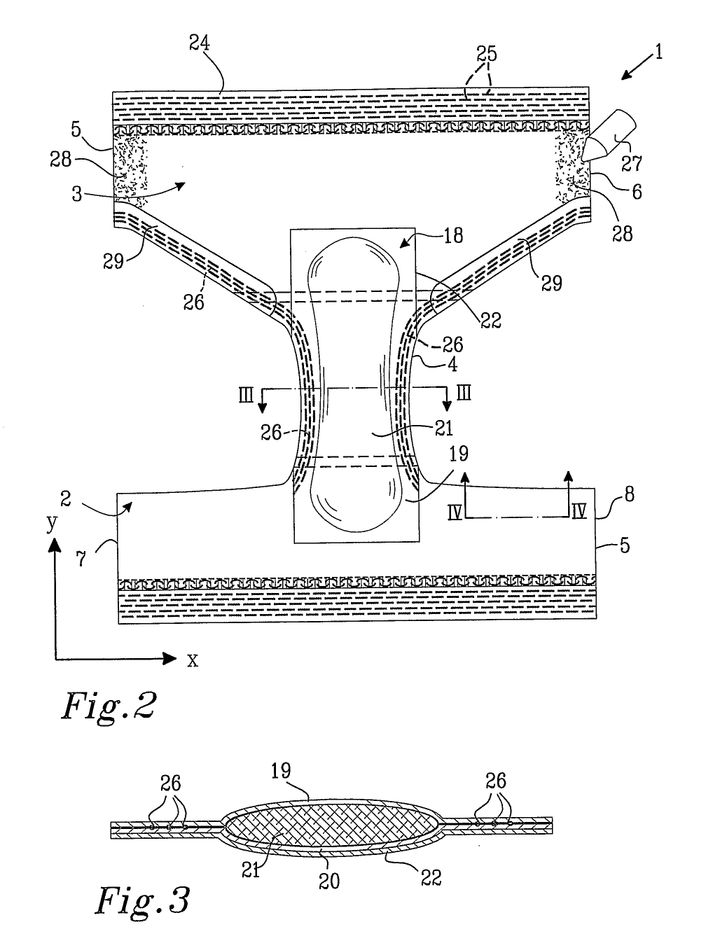

The elastic web material constitutes the sole component of at least parts of the front and back panels 2 and 3. In at least 20%, preferably at least 25%, more preferably at least 30% and most preferably at least 40% of the total surface area of the article, as seen in a flat state according to FIG. 2 the elastic web material 14 constitutes the sole component of the front and back panels.

No additional elasticized side panels joining the front and back panels 2 and 3 are needed when using the elastic web material 14.

It is further desired that the elastic web material has a puncture resistance of at least 15N as measured according to ASTM Designation D3763-02. Preferably, the elastic web material of the present invention has a puncture resistance of at least 20N, and more preferably at least 30N.

The elastic web material may preferably have a softness according to Kawabata of at least 20, preferably at least 30 and most preferably at least 40. It is further desired that it has a formability according to Kawabata of no more than 50, preferably no more than 30, more preferably no more than 20 and most preferably no more than 10. It is also desired that the elastic web material has a drapability according to Kawabata of no more than 40. The softness, formability and drapability according to Kawabata are measured according to the test methods given in PCT/SE2004/001004.

The bonds between elastic web materials 14 of the type described above and of other types of elastic and inelastic thin web materials sometimes are insufficient. Examples of such bonds are in the side seams 9 and 10, which are exerted to considerable stresses when being pulled on over the hips of the-wearer. The bonds in these side seams 9 and 10 are normally accomplished by ultrasonic welding or thermobonding, wherein a bonding pattern is formed.

According to this invention the bonds, such as in the side seams 9 and 10 have been reinforced by applying to at least one of the elastic web materials 14 an additional material prior to bonding, said additional material is non-tacky at a temperature of 20° C. It is preferred that the additional material is already non-tacky at a temperature of 30° C., preferably also at 40° C. and more preferably also at 60° C. The additional material is applied in at least a part of the area to be bonded. The problem of handling adhesive materials that are tacky also below their application temperature is herewith avoided. The presence of the additional material in the area to be bonded improves the bonding strength of the bond between the two elastic web materials 14.

The additional material is applied at a temperature at which it is at least partly molten or softened so that it will adhere to the web material to which it is applied. One preferred way of applying the additional material is by means of spraying. A spray equipment is schematically shown in FIG. 2 and is designated with the numeral 27. The additional material is preferably applied in the form of discontinuous fibers 28 of a limited length or continuous filaments by known spray application techniques.

The additional material, for example fibers 28, preferably comprises a polymeric material selected from polyolefins including polyethylene and polypropylene, polyesters, polyamides, copolymers and mixtures of such polymeric materials.

An alternative way of applying the additional material is by coating, slot coating, printing, embossing and similar known application techniques.

The elastic web material 14 preferably also contains a polymeric material selected from polyolefins including polyethylene and polypropylene, polyesters, polyamides, copolymers and mixtures of such polymeric materials.

The elastic web material 14 may be in a stretched condition during bonding. It may for example be stretched to a length, in longitudinal and/or transverse direction, which exceeds its untensioned length with at least 20% during bonding. In such case the need for reinforcing the bond is accentuated, since when in a stretched condition, the elastic web material is thinner which gives a weaker bond, as when in a non-stretched condition.

The additional material 28 may be applied to only one of the web materials to be bonded or to both. The additional material 28 may be applied over the entire area of a web material to be bonded or only to selected areas thereof. It may also be applied to a larger area than the one to be bonded in order to ensure that the additional material is applied to an area that is large enough to provide the desired bonding strength. Since the additional material after application solidifies to a non-tacky state its presence outside the bonded area, for example the side seams 9 and 10, does not disturb the further manufacturing process nor impair the product quality, as for example application of an adhesive outside the bonded area would do. The additional material may further be applied either between the web materials to be bonded together, i.e. on the inside of the seam between the web materials, or on the outside thereof.

In the case of the embodiment shown in the drawing the additional material 28 may be applied along the entire side seams 9 and 10 or only in selected portion thereof, for example the portion outside the waistband 24, which comprises more material layers which may provide a sufficient bonding strength. The additional nonwoven strip 29 that is present in the leg opening area may also provide a reinforcing effect on the bond strength, so in a further embodiment the additional material 28 is applied only in the central area of the side seam 9 and 10.

In other embodiments of pant diapers the additional material 28 is applied only in the areas adjacent the waist opening and the leg openings, in which the stresses exerted to the side seams are most critical.

The tensile strength of the side seams (9, 10), in a direction transverse (x) to the side seam, is at least 5 N/25.4 mm, preferably at least 7 N/25.4 mm and more preferably at least 9 N/25.4 mm at least in the portions reinforced by the additional material (28).

The additional material 28 may of course be applied also in other areas to be bonded, such as in the area where the elastic waistband 24 is bonded to the body panels 9 and 10 and/or in the area where the elastic web material 14 is bonded to the crotch portion web material 22.

The additional material 28 is applied in a basis weight of at least 10 gsm, preferably at least 20 gsm and more preferably at least 30 gsm.

It is understood that the present invention includes all embodiments in which two web materials, elastic or inelastic, are bonded to each other by applying thermal energy or ultrasonic energy thereto. It especially refers to bonding web materials together that are components of a personal care absorbent article. According to one aspect of the invention the web materials have a basis weight of no more than 100 gsm, preferably no more than 90 gsm.

### EXAMPLE

An elastic laminate material comprising an inner apertured three-layer elastic film of PE-SEBS-PE, basis weight 36 g/m² and two outer layers of spunbond material, PP (polypropylene), each having a basis weight of 22 g/m². The laminate is produced by a modified version of the method disclosed in WO 03/04788, wherein one spunbond layer is applied to the film in a tacky state and will thus bond to the film layer, while the other spunbond layer is adhesively laminated to the film layer using for example a pressure sensitive hot melt adhesive (glue amount 3 g/m²). The laminate is incrementally stretched, at which the non-elastic spunbond layers are stretched to a point below the elongation at maximum load to retain some strength in the spunbond layers. The elasticity of the laminate after stretching is close to the elasticity of the elastic film layer.

The above-mentioned basis weights of the layers refer to the finished laminate after stretching. Before stretching the basis weight of the individual layers were: inner film layer 40 g/m², outer spunbond layers 25 g/m² each and glue layer 3 g/m². Since it is difficult to measure the basis weights of the individual layers after lamination and stretching an approximation has been made from the basis weights of the layers before lamination and stretching. The laminate before stretching had a total basis weight before stretching of 93 g/m² and after stretching it had a basis weight of 85 g/m², which means a deformation of about 10%. It is then assumed that the deformation of the individual fibrous layers and the film layer is the same, i.e. about 10%.

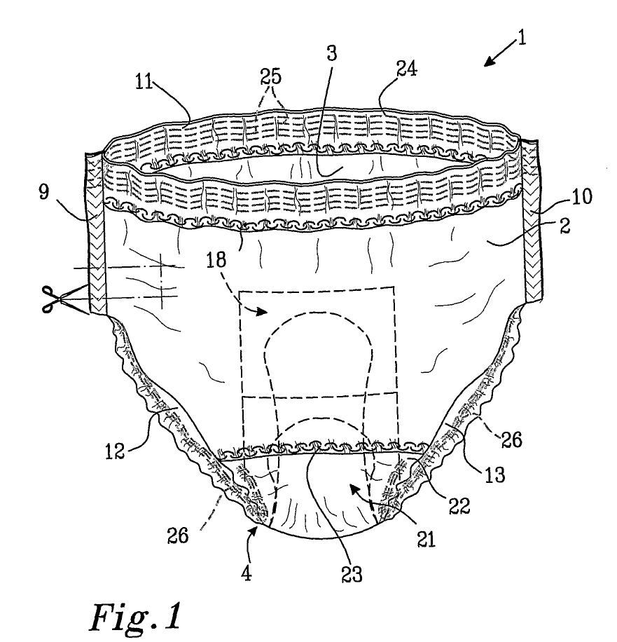

Pant diapers as disclosed above and as shown in FIGS. 1-3 were produced comprising the elastic laminate as front and back body panels. The side seams between the front and back body panels were in some of the products reinforced by spray application of fibers of polypropylene, Borealis HL612FB. The application temperature for the polymer was between 220 and 250° C. and the spray head was held between 20 and 50 mm from the elastic laminate material to be coated. The fibers were either sprayed between the material webs in the side seam or on the outside of thereof. Tensile strength tests of the side seams were performed and reported below.

### Elasticity Test

The method measures how an elastic material behaves at repeated load and unload cycles. The sample is stretched to a predetermined elongation and a cyclic movement between 0 and said predetermined elongation is performed. Desired load and unload forces are recorded. The permanent, i.e. remaining, elongation of the relaxed material is measured.

A tensile tester, Lloyd LRX, able to perform cyclic movements and equipped with a printer/plotter or software presentation is used. The sample is prepared by cutting it to a width of 25 mm and a length that is preferably 20 mm longer than the distance between the clamps in the tensile tester.

The tensile tester is calibrated according to the apparatus instructions. The parameters needed for the test (load and unload forces) are adjusted to:

| | | |
|---|---|---|
| Crosshead speed: | 500 | mm/min |
| Clamp distance: | 50 | mm |
| Preload: | 0.05 | N |

The sample is placed in the clamps according to the marks and it is made sure that the sample is centered and fastened perpendicularly in the clamps. The tensile tester is started and three cycles between 0 and the predetermined elongation, equal to the highest defined 1^{st} load, are performed. Before the last cycle, the sample is relaxed for 1 minute, then the permanent elongation is measured by stretching the sample until a force of 0.1 N is detected and the elongation is read.

The permanent elongation after relaxation should be less than 10% and is measured by the method above. Thus an elasticity of 30% is defined as that the laminate should have a permanent relaxation after elongation of less than 10% after being exerted to an elongation of 30% in the tensile tester above. An elongation of 30% means an elongation to a length that is 30% longer than the initial length of the sample.

### Tensile Strength of Side Seams

The tensile strength of the side seams is measured according to the ASTM D 882 method described in the patent application PCT/SE2004/001004. Test specimens are cut from the products, as shown in FIG. 1, in areas which are reinforced with the additional material. The width of the test specimens were 25.4 mm and the length, if possible, 50 mm longer than the distance between the clamps of the tensile test instrument Instron 4301. FIG. 5 and 6 illustrate how the test specimens are secured in the clamps.

To illustrate the effect of the reinforcement of the side seam by the application of additional material, Table 1 shows the test results for measurements of the tensile strength of a side seam (ultrasonically welded) with and without reinforcement with additional material in the form of sprayed fibers of polypropylene, Borealis HL612FB. In sample 1 the additional material was sprayed on the outside of the side seam while in sample 2 it was sprayed on the inside of the side seam between the joined elastic laminate materials. In the reference material no additional material was applied to the side seam. The values given in Table 1 are mean values from 10 measurements.

TABLE 2

| | Force at 5% | Force at 10% | Force at 25% | Max. | Elongation |
|---|---|---|---|---|---|
| | Elongation | Elongation | Elongation | force | at Max. |
| Sample | (N) | (N) | (N) | (N) | load (%) |
| Sample 1 | 0.27 | 0.58 | 1.32 | 16.2 | 117 |
| Sample 2 | 0.29 | 0.57 | 1.35 | 17.5 | 115 |
| Reference | 0.19 | 0.43 | 1.06 | 15.1 | 127 |

It is understood that although the invention has been described with reference to preferred embodiments, several modifications are possible within the scope of the claims. The invention therefore intends to cover any variations or equivalents which are within known or customary practice within the technical field to which it belongs.

1. A method of bonding a first web material (2) and a second web material (3) to each other, wherein the two web materials are brought together in the area to be bonded and applying thermal energy or ultrasonic energy to at least one of the web materials in the area to be bonded, characterized in that prior to bringing the two web materials together an additional material (28) that is non-tacky at a temperature of 20° C. is applied to at least one of the web materials in at least a part of the area to be bonded, said additional material (28) is applied at a temperature at which it is at least partly molten or softened so that it will adhere to the web material (2, 3) to which it is applied.

2. The method as claimed in claim 1, characterized in that said additional material is applied by means of spraying (27).

3. The method as claimed in claim 2, characterized in that said additional material (28) is applied in the form of fibers or continuous filaments.

4. The method as claimed in any of the preceding claims, characterized in that the area to be bonded amounts to less than 10% of the total surface area of each of said first and second web materials (2, 3).

5. The method as claimed in claim 4, characterized in that the area to be bonded has a defined length and width, wherein said width is no more than 30 mm, preferably no more than 25 mm and preferably no more than 20 mm.

6. The method as claimed in any of the preceding claims, characterized in that the web material (2, 3) comprises a polymeric material selected from polyolefins including polyethylene and polypropylene, polyesters, polyamides, copolymers and mixtures of such polymeric materials.

7. The method as claimed in any of the preceding claims, characterized in that said additional material (28) comprises a polymeric material selected from polyolefins including polyethylene and polypropylene, polyesters, polyamides, copolymers and mixtures of such polymeric materials.

8. The method as claimed in any of the preceding claims, characterized in that said additional material (28) is non-tacky at a temperature of 30° C., preferably also at 40° C. and more-preferably also at 60° C.

9. The method as claimed in any of the preceding claims, characterized in that said first and second web materials (2, 3) are components in a personal care absorbent article.

10. The method as claimed in claim 9, characterized in that said first and second web materials form part of first and second body panels (2, 3 ) of a pant-type absorbent article (1) and that said area to be bonded of said web materials intended to form a side seam (9, 10) of the pant-type absorbent article.

11. The method as claimed in any of the preceding claims, characterized in that at least one of said first and second web materials (2, 3) comprises an elastic web material.

12. The method as claimed in claim 11, characterized in that both the first and second web materials (2, 3) are elastic web materials.

13. The method as claimed in claim 11 or 12, characterized in that the first and/or second web materials (2, 3) are elastic laminates.

14. The method as claimed in any of claims 11-13, characterized in that the elastic web material is in a stretched condition during bonding.

15. The method as claimed in claim 14, characterized in that the elastic web material is stretched to a length which exceeds its untensioned length with at least 20% during bonding

16. The method as claimed in any of claims 10-15, characterized in that said additional material is applied to selected areas of said side seam (9, 10).

17. The method as claimed in any of claims 11-16, characterized in that the elastic web material has a basis weight of no more than 100 g/m², preferably no more than 90 g/m².

18. The method as claimed in any of claims 11-17, characterized in that the elastic web material is an elastic laminate (14) comprising an elastic film (17) applied between layers (15, 17) of fibrous material.

19. The method as claimed in any of claims 11-18, characterized in that the elastic web material constitutes the sole component of the first and second body panels (2, 3) at least in selected portions of the areas which are bonded together to form said side seams (9, 10).

20. The method as claimed in any of claims 11-19, characterized in that the elastic web material has an elasticity in the transverse direction of the article of at least 30%, preferably at least 50%, more preferably at least 70%, when measured according to the elasticity test specified in the description.

21. The method as claimed in any of the preceding claims, characterized in that the additional material (28) is applied in an amount corresponding to a basis weight of at least 10 gsm, preferably at least 20 gsm and more preferably at least 30 gsm.

22. A personal care absorbent article comprising an absorbent structure and first and second web materials joined together by thermal bonding or ultrasonic frequency bonding to form a bonded area, characterized in that an additional material (28) that is non-tacky at a temperature of 20° C. is present in at least a part of said bonded area, said additional material (28) having been applied at a temperature at which it is at least partly molten or softened so that it adheres to at least one of the web materials.

23. The absorbent article as claimed in claim 22, characterized in that said additional material (28) is in the form of fibers or filaments.

24. The absorbent article as claimed in claims 22 or 23, characterized in that the area to be bonded amounts to less than 10% of the total surface area of each of said first and second web materials (2, 3).

25. The absorbent article as claimed in claim 24, characterized in that the area to be bonded has a defined length and width, wherein said width is no more than 30 mm, preferably no more than 25 mm and preferably no more than 20 mm.

26. The absorbent article as claimed in any of claims 22-25, said article being a pant type absorbent article comprising first and second body panels (2, 3) and an intermediate crotch portion (4) provided with a pair of leg openings (12, 13), said first and second body panels being boded together to form side seams (9, 10) of the pant article, characterized in that said first and second web materials form at least part of said first and second body panels (2, 3) and that said bonded area is a side seam (9, 10) of the pant-type absorbent article.

27. The absorbent article as claimed in any of claims 22-26, characterized in that at least one of said first and second web materials (2, 3) comprises an elastic web material.

28. The absorbent article as claimed in claim 27, characterized in that both of said first and second web materials (2, 3) comprises an elastic web material.

29. The absorbent article as claimed in claim 27 or 28, characterized in that that the elastic web material has a basis weight of no more than 100 g/m², preferably no more than 90 g/m².

30. The absorbent article as claimed in any of claims 27-29, characterized in that the elastic web material is an elastic laminate (14) comprising an elastic film (17) applied between layers (15, 17) of fibrous material.

31. The absorbent article as claimed in any of claims 26-30, characterized in that said additional material is applied to selected areas of said side seam (9, 10).

32. The absorbent article as claimed in any of claims 27-31, characterized in that the elastic web material constitutes the sole component of the first and second body panels (2, 3) at least in selected portions of the areas which are bonded together to form said side seams (9, 10).

33. The absorbent article as claimed in any of claims 27-32, characterized in that the elastic web material has an elasticity in the transverse direction of the article of at least 30%, preferably at least 50%, more preferably at least 70%, when measured according to the elasticity test specified in the description.

34. The absorbent article as claimed in any of claims 22-33, characterized in that the additional material (28) is present in an amount corresponding to a basis weight of at least 10 gsm, preferably at least 20 gsm and more preferably at least 30 gsm.

35. The absorbent article as claimed in any of claims 22-34, characterized in that the web material (2, 3) comprises a polymeric material selected from polyolefins including polyethylene and polypropylene, polyesters, polyamides, copolymers and mixtures of such polymeric materials.

36. The absorbent article as claimed in any of claims 22-35, characterized in that the additional material (28) comprises a polymeric material selected from polyolefins including polyethylene and polypropylene, polyesters, polyamides, copolymers and mixtures of such polymeric materials.

37. The absorbent article as claimed any of the claims 26-36, characterized in that the tensile strength of the side seams (9, 10), in a direction transverse (x) to the side seam, is at least 5 N/25.4 mm, preferably at least 7 N/25.4 mm and more preferably at least 9 N/25.4 mm at least in the portions reinforced by the additional material (28).
